(19) United States
(12) Patent Application Publication
(10) Pub. No.: US 2020/0037910 A1
(43) Pub. Date: Feb. 6, 2020

(54) INJECTABLE HYDROGEL-FORMING POLYMER SOLUTION FOR A RELIABLE EEG MONITORING AND EASY SCALP CLEANING

(71) Applicants: UNIVERSIDADE DO PORTO, Porto (PT); TECHNISCHE UNIVERSITAT ILMENAU, Ilmenau (DE)

(72) Inventors: José Carlos MAGALHÃES DUQUE DA FONSECA, Porto (PT); Paulo Eduardo TEIXEIRA BATISTA PEDROSA, Porto (PT); Patrique FIEDLER, Ilmenau (DE); Ana Cristina OLIVEIRA MARTINS, Porto (PT); Jans HAUEISEN, Ilmenau (DE)

(21) Appl. No.: 16/338,478

(22) PCT Filed: Sep. 29, 2017
(86) PCT No.: PCT/IB2017/056012

§ 371 Date: Mar. 31, 2019

(30) Foreign Application Priority Data
Sep. 30, 2016 (PT) 109647

(51) Int. Cl. A61B 5/0476; C08J 3/075; C08K 3/30

(52) U.S. Cl. CPC A61B 5/0476; C08J 3/075; C08K 2003/3045; A61B 2562/14; C08J 2305/04; C08K 3/30

(57) ABSTRACT

An injectable composition is described, which is capable of forming an hydrogel for electroencephalography (EEG) recording. The obtained hydrogel and method for its production is also an object of the invention, as well as the use of the injectable composition for EEG recording.

The injectable composition comprises: natural or synthetic polymers, preferably alginate; a polymerization initiation system or a cross-linking agent, preferably calcium salts; and at least one ionized salt. The injectable composition can be applied into the electrode cavities of common commercial EEG caps and forms a solid hydrogel shortly after application. When the cap is taken off, the hydrogel either remains inside the electrode cavities, or it breaks into parts that are easily removed from the hair with a comb. It allows a faster and easier cleaning, reduces movement artefacts and also the risks of electrodes short-circuiting due to gel running, hence increasing EEG data reliability.

### FIELD OF THE INVENTION

The present invention relates with electrolytic gels used to interface the silver/silver-chloride (Ag/AgCl) electrode with the skin. The injectable hydrogel-forming composition allows gelation shortly after application, ensuring a reliable electrical contact for the electrophysiological signal acquisition. More specifically, the electrolytic gel of the present invention is particularly useful in the field of EEG recording.

### BACKGROUND OF THE INVENTION

The study of brain through the monitoring of bioelectric potential fluctuations on the scalp surface dates back to the first half of the twentieth century. From the technical point of view the recording of the so-called EEG signals involves an ionic-to-electronic signal transduction that takes place at the interface between the bio-electrode and the electrolytic gel, which allows the signal to be acquired and processed by the electronic equipment. The Ag/AgCl electrodes, commonly applied with an electrolytic gel to reduce contact impedance, have been the gold standard for EEG for many years due to their reliability, low level of intrinsic noise and electric potential stability [1,2]. EEG has been systematically used in the investigation of physiological conditions and pathologies of the human brain, e.g. stroke [3] and epilepsy [4]. In the last decade the application of the technique has been extended to the study of the human brain function in non-clinical applications such as sports sciences [5] and brain computer interfaces [6].

On the other hand, the actual Ag/AgCl/electrolytic gel combination has been the source of many problems. Indeed, there is a non-negligible risk of electrode short-circuits due to gel running and spreading, particularly in high density EEG (128 to 256 electrodes). Furthermore, the gel strongly sticks to the hair and scalp, forcing the patient to thoroughly wash the head after the exam to remove the gel residues.

The need for a more expeditious EEG system, combining the performance of the actual Ag/AgCl electrodes electrolytic gel combination with a faster and easier application/removal protocol, has translated into a high number of technical solutions [7-12]. The most relevant technical approaches in the framework of the disclosed invention will be analyzed next.

A long-time candidate to replace the Ag/AgCl electrode is the so-called “dry” electrode. A dry electrode makes use of an inert, conductive material that mechanically couples with the skin for signal transfer, dispensing with the use of electrolytic gels and thus forming the ideal plug-and-play system [7,13,14]. However, the interfacial impedance is substantially higher, making essential the integration of a pre-amplification stage on the electrode [13,14] or the use of active shielding for signal transmission [15]. Dry electrodes proved to be more susceptible to movement artefacts and the contact impedance is strongly dependent on the electrode adduction pressure [16].

A different approach that enables a low electrode/scalp contact impedance in the absence of a gel contact is using a micro-needles array based electrode that perforates the stratum corneum (SC) highly insulating skin layer [8]. Since the SC is short-circuited the performance of these electrodes is close to that obtained with commercial Ag/AgCl electrodes and gel. However, 5% of the spikes were reported to break during the exam and remained embedded in the epidermis, thus increasing the risks of infection and inflammatory reactions.

A further alternative to obtain a low impedance (wet) scalp contact consists in using the working principle of the felt pen. In this case the electrodes are formed by a wick material (felt pen tip) and have a liquid reservoir on the back. The material can be either a polymer [17], a metal [18] or a ceramic [19], whose capillarity properties enable it to dispense a moisturizing liquid and consequently maintain a wet electrode/skin interface without dirtying the scalp. The results proved the viability of the concept and the long term autonomy of the device (up to 8 h), but the presence of the liquid and the fact that these are usually quite complex multi-part devices, may increase the costs and raise functional problems during repetitive applications with regard to cleaning and mechanical stability.

Hydrogels have also been successfully used to produce biocompatible, compliant and ionically conductive electrode/scalp interfaces, their application being very common in ECG and EMG disposable electrodes [2].

A few works also exist on the application of hydrogels to the area of EEG recording. Alba et al [20] reported about a polyacrylate hydrogel swollen with a humectant solution (to increase skin conductivity) to establish the scalp interface, with an Ag/AgCl wire sensor embedded in the hydrogel for signal transduction. In-vivo testing was performed with a single electrode and showed that the impedance of the hydrogel/scalp contact lied between those of “wet” and “dry” electrodes, it remained stable for up to 8 h and basic EEG signals could be recorded in good quality. This work can be seen as a proof of concept of hydrogels application for EEG electrode fabrication. Kleffner-Canucci et al [21] used an N-isopropyl acrylamide co-acrylic acid (NIPAm) hydrogel dissolved in a saline solution as a gel replacement product to increase the EEG recording time. The product was tested in a multi-electrode array with the Geodesic Sensor Net (GSN) caps (Electrical Geodesics, Inc, USA). It was demonstrated that the new formulation decreases the water evaporation rate, allowing extended EEG recording durations, up to 4.5 h.

In commercial terms the most common setup for EEG consists of a cap with an embedded Ag/AgCl multiple electrode array, where each electrode site has an associated cavity that is filled with the electrolytic gel before application, to bridge the electrode to the scalp. Thus, the electrode doesn't touch the scalp. Different realizations of this solution are available, such as the Waveguard (ANT, Medical Imaging Solutions GmbH), the Quik-Cap (Compumedics—Neuro Scan), the BioSemi (BioSemi B.V., the Netherlands) or the EasyCap (EasyCap GmbH) systems. Electrical Geodesics Inc. proposes a different system, the GSN net, where the electrodes are connected through a geodesics net and each electrode site has a sponge that is swollen with a saline solution just before the exam, thus avoiding the use of the gel. The swelling is performed by simply dipping the GSN net in a salt solution before the exam. The main advantages against the gel based systems are the much shorter preparation time and the fact that, at the end, the hair doesn't need to be washed. Besides the drying effects during longer acquisitions, the main disadvantage of the approach of sponges+saline solution are unavoidable electrical shortcuts between multiple electrodes. This considerably limits applicability to well-defined fields and must always be considered in signal processing. For long term monitoring an electrolytic paste or a special electrolyte can be used instead of the saline solution. The Neuroelectrics Company Inc. proposes a hydrogel based approach with the Solidgeltrode®, which includes a “solid gel” part that is sold as a consumable and fits to the electrode cavity, bridging the electrode to the scalp. Also in this case there is no need to wash the head after the exam.

### SUMMARY OF THE INVENTION

This invention relates with a polymer-based electrolyte that is used to bridge Ag/AgCl EEG electrodes to the scalp. An injectable polymeric composition is described, which is capable of forming an hydrogel for EEG recording. The obtained hydrogel and method for its production is also an object of the invention, as well as the use of the injectable composition for reliable EEG monitoring and easy scalp cleaning.

The injectable hydrogel-forming polymeric composition comprises: natural or synthetic polymers, preferably alginate; a polymerization initiation system or a cross-linking agent, preferably calcium salts; and at least one ionized salt to provide adequate electrical conductivity. The hydrogel viscosity can be adjusted by varying the alginate concentration and the gelation rate may be tuned by varying the alginate to calcium salts ratio. Similarly to many commercial EEG electrolytic gels, the product is injected in a state of low viscosity into the electrode cavities built in commercial electrode caps. However, unlike the common electrolyte gels for EEG applications, the new formulation undergoes gelation shortly after application, forming a solid hydrogel structure that embeds the hair layer and reliably bridges the Ag/AgCl electrode to the scalp. The presence of ionized salts enables the EEG biosignal conduction from the scalp to the electrode and the presence of a skin permeation enhancer helps to lower the skin impedance. The main advantage of the proposed hydrogel product against common electrolytic gels is that, after the end of the EEG recording the hydrogel comes off with the cap or breaks into parts that are easily removed with a comb. Conversely, the normal gel spreads and sticks to the hair and scalp and requires a hair wash to be removed. Moreover, an important technical advantage over normal electrolytic gels is that, since a solid product is formed shortly after application, the risk of gel running away from the application point, short-circuiting neighboring electrodes, is substantially reduced. This is particularly important for high density EEG applications where the number of electrodes can reach 128 or 256. On the other hand, as only skin approved agents are used to ensure skin permeation, this hydrogel is less susceptible to cause allergic reactions.

### BRIEF DESCRIPTION OF DRAWINGS

Further characteristics and advantages of the injectable composition according to the present invention will be more apparent from the following description of some embodiments thereof, made as a non-limiting examples, with reference to the appended drawings wherein:

FIG. 1 shows the gelation time (three repetitions) of the proposed hydrogels plotted as a function the calcium sulfate-to-alginate ratio. The inset picture shows the final shape and uniformity of the gels after complete gelation.

FIG. 2 shows the measurement setup for the simultaneous EEG acquisition using conventional electrolyte paste (grey) and hydrogel (black): a) overall scheme of the parallel measurement setup and b) equidistant electrode arrangement indicating compared adjacent electrodes (connected by lines).

FIG. 3 shows the time domain overlay plot of exemplary adjacent channels and EEG sequences of 6 sec. length: a) EEG containing eye blinks recorded using channels LL1 (conventional paste) and LD1 (hydrogel); b) resting state (0-3 sec.) and alpha activity (3-6 sec.) EEG recorded using channels LL13 (conventional paste) and LL12 (hydrogel).

FIG. 4 shows the grand average over all 3 volunteers of the visual evoked potential (VEP) tests: a), c), e) Ag/AgCl electrodes in combination with conventional electrolyte paste; b), d), f) Ag/AgCl electrodes in combination with hydrogel; a), b) Butterfly plot of all channels without artefacts; c), d) global field power (GFP) calculated over all channels without artefacts; e), f) topographic potential mappings of the respective N75 and P100 components.

FIG. 5 shows the grand average over all 3 volunteers and 64 channels of the welch estimation of the power spectral density (PSD): Solid lines indicate the PSD of EEG containing eminent alpha activity while dotted lines indicate the PSD of resting state EEG.

FIG. 6 shows photographs of different head positions of two volunteers after taking off the cap: a) right fronto-temporal position: hydrogel easily comes off (black circles) while conventional electrolyte paste needs extensive cleaning (white circles); and b)-d) CP1 head positions: after the removal of the cap, b) the fully gelled hydrogel can be easily removed with a comb and c) the hairy position is easily and completely clean d) after 10 s with a dry towel, no washing.

### DETAILED DESCRIPTION OF THE INVENTION

It is an object of the present invention an injectable hydrogel-forming polymeric composition that is capable of forming a hydrogel for reliable EEG monitoring and easy scalp cleaning, said composition comprising: a first component, selected from the group consisting of natural and synthetic polymers; and a second component, selected from the group consisting of a polymerization initiation system or a cross-linking agent.

In a preferred embodiment, the first component is a solution comprising alginate, and the second component is a solution comprising calcium salts.

In a more preferred embodiment, the first component further comprises at least one ionized salt in a concentration ranging from 0.1% to 10% to provide adequate electrical conductivity.

In another preferred embodiment, the first component further contains a humectant, preferably glycerol or propylene glycol, and a skin penetration enhancer, preferably Tween®80.

In a more preferred embodiment, the first component is a solution comprising 2.8% (w/v) sodium alginate, 6% (v/v) Tween®80, 10% (v/v) propylene glycol and 1.8% (w/v) sodium chloride; and the second component is a solution comprising 0.34% (w/v) calcium carbonate, 0.14% (w/v) calcium sulfate dehydrate and 1.18% (w/v) gluconolactone.

It is also an object of the present invention an hydrogel for reliable EEG monitoring and easy scalp cleaning, formed of said injectable hydrogel-forming.

In a preferred embodiment, the gelation rate of said hydrogel is adjustable by changing the alginate to calcium salts ratio.

In another preferred embodiment, the viscosity of said hydrogel is adjustable by varying the alginate concentration.

In an even more preferred embodiment, said hydrogel is suitable for application on the cavities of the EEG electrode.

It is also an object of the present invention a method of producing a hydrogel for reliable EEG monitoring and easy scalp cleaning comprising: mixing the first component and the second component as described above.

It is also an object of the present invention the use of an injectable hydrogel-forming composition comprising the following steps:

i) providing a first component and a second component as described above;

ii) joining the first and second component to induce hydrogel production and applying into the electrode cavities of the EEG cap system;

iii) removing the EEG cap after EEG recording with attached solid hydrogel;

iv) cleaning the solid hydrogel pieces from the hair with a comb if necessary.

In a preferred embodiment, in step ii) the first and the second components are mixed before application.

In an even more preferred embodiment, in step ii) a double syringe equipped with a mixer nozzle is used to lower gelation time.

The herein disclosed invention thus describes the composition and application procedure of a hydrogel-forming formulation that is intended to advantageously replace the traditional electrolytic gels and pastes used for EEG recording. In addition, the electrolytic gel herein presented can be applied into the electrode cavities of common commercial EEG caps and helmets. The formulation of the gel can be presented in the form of one or two components. In the first case gelation is triggered by supplying energy in the form of heat of light of defined adequate wavelength, whereas in the second case the two components are mixed to form the hydrogel. Most often one of the components will be a monomer, a macromer or a polymer and the second component will contain a polymerization initiation system or a cross-linking agent.

The hydrogel includes at least one ionized salt in a concentration ranging from 0.1% to 10% to provide an adequate electric conductivity and, in addition, it may also contain a humectant, such as glycerol or propylene glycol, and a skin penetration enhancer in order to help hydrating the stratum corneum insulation layer and make it more permeable. In any case the interference of any foreign chemical agents with the gelation kinetics must be duly assessed, as well as the biocompatibility of the products.

A preferred formulation includes a solution containing 2.8% (w/v) sodium alginate, 6% (v/v), Tween® 80, 10% (v/v) propylene glycol, 1.8% (w/v) sodium chloride and a second solution containing 0.34% (w/v) calcium carbonate, 0.14% (w/v) calcium sulfate dehydrate and 1.18% (w/v) of gluconolactone. The solutions are mixed in equal parts to start the gelation process. The gelation rate can be adjusted by changing the alginate to calcium salts ratio. The viscosity of the initial solution can be adjusted by varying the alginate concentration.

In the present invention, the application of the hydrogel in its initial low-viscosity state into the electrode cavities is performed with a syringe. In the case where the formulation consists of two components the mixture can be prepared before application, for example by shaking the two components in a plastic container filled with stainless steel spheres to facilitate the mixture. Depending on the number of electrodes to fill, and due to the defined pot life of the product more than one batch of the product may have to be prepared (a solid is formed preventing the injectability). An adequate gelation time for many applications may be 8-10 minutes. Therefore, the formulation should preferentially be applied by using a double syringe equipped with a mixer nozzle. In this case the gelation time can be lowered to about 3-5 minutes, which is enough for the gel components to mix in the nozzle and spread around the hair inside the electrode cavities.

Once the exam has finished the cap should be removed as if the regular electrolytic gel was used. However, the hydrogel will either stay attached to the electrode cups, or it will break into parts that can be easily removed with a comb. In contrast to other alternatives, the hydrogel will be easily removed but the cleaning procedure should be carried out while the hydrogel is swollen with water.

When compared with the existent technical solution proposed by Electrical Geodesics with the GSN cap, both approaches dispense with the hair wash after the EEG exam. However, in spite of a shorter electrode preparation time, with the Electrical Geodesics approach the risk of electrode short-circuits is even higher than in case of conventional electrolytic gels due to the presence of the saline solution in the sponges. Kleffner-Canucci [22] approach also starts with a hydrogel based electrolyte but, according with the authors, this is intended to fixate the water thus reducing the electrolyte evaporation rate and extending the EEG recording time. Nothing is said about the possibility of the NIPAm electrolyte gelation during the EEG acquisition, therefore it is to believe that this gel behaves as a normal electrolytic gel from the point of view of its behavior in contact with the hair and scalp.

The so-called Solidgeltrode® electrode system marketed by Neuroelectrics was proposed with the same declared goal of the present invention: to achieve clean hair and scalp after the EEG exam, for which the company proposes to use a hydrogel. However, instead of using a solution that is injected into the electrode cavities to form the hydrogel, the company already sells the hydrogel, which fits a specific electrode cavity of Neuroelectrics cap. It follows that the technical solution of our invention is much more flexible as it can be used with any cap system and electrode material. On the other hand, from the technical side, when the Solidgeltrode® system is used in patients with dense hair, or strongly curled hair, it will be difficult to make the already solid hydrogel part penetrate the hair and reach the scalp to form a reliable contact during the exam. In the case of the present invention the gel solution is injected in the liquid form, thus being able to make a continuous path through the hair and reach the scalp. Once the hydrogel is formed the hair will help maintaining the scalp contact.

### Example

In order to demonstrate the ability of the hydrogel-forming electrolyte concept to replace the traditional EEG electrolytic gel, a sodium alginate polymer (Sigma Aldrich, MI, USA, ref. 71238) and two calcium sources (gelation promoters) were chosen. The possibility of tuning the gelation time was studied by preparing several solutions with different compositions, showing that the gelation rate may be adjusted by varying the alginate-to-calcium ratio Table I.

TABLE I
Composition of the produced hydrogels

| | [alginate] | [PG] | [Tween 80] | [GDL] | [CaCO₃] | [CaSO₄•2H₂O] | [NaCl] |
|---|---|---|---|---|---|---|---|
| Hydrogel | w/v (%) | v/v (%) | v/v (%) | w/v (%) | w/v (%) | w/v (%) | w/v (%) |
| H1 | 1 | 5 | 3 | 0.33 | 0.09 | 0.09 | 0.9 |
| H2 | 1.4 | 5 | 3 | 0.46 | 0.13 | 0.13 | 0.9 |
| H3 | 1.4 | 5 | 3 | 0.59 | 0.17 | 0.07 | 0.9 |
| H4 | 1 | 5 | 3 | 0.62 | 0.17 | 0.07 | 0.9 |

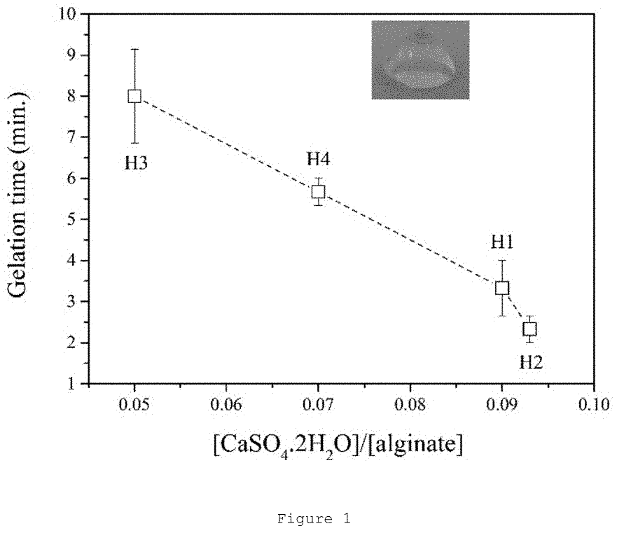

It is also possible to include a preservative agent to the formulation. FIG. 1 shows the correlation between calcium sulfate: sodium alginate ratio and the gelation time.

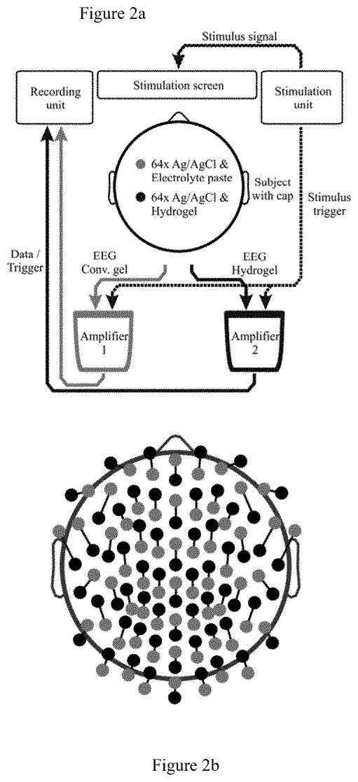

The proof of concept was performed by using the H3 formulation and a 128 electrodes Waveguard cap (ANT B.V., Netherlands). The preliminary in-vivo EEG tests were performed on three healthy adult volunteers. A simultaneous measurement setup was applied allowing for parallel acquisition of EEG data using two independent sets of 64 identical Ag/AgCl electrodes in combination with the commercial electrolyte paste (ECI Electro-Gel) and the selected hydrogel. The measurements took place after full gelation of the hydrogel. The overall measurement setup and electrode arrangements are shown schematically in FIG. 2a and FIG. 2b, respectively.

Before the start and after completion of the EEG recordings, the electrode-skin impedances at all electrode positions were measured using the integrated impedance measurement function of the EEG amplifier using a square signal of 8 Hz frequency and a 50 percent duty cycle. The mean electrode-skin impedance, calculated over all volunteers and channels, decreased from 17±16 kΩ to 12±5 kΩ for the conventional paste, and 31±20 kΩ to 25±17 kΩ for the hydrogel. The decreasing values and the variation of both impedances indicate the hydration effect of both the paste and the hydrogels on the scalp. The higher impedance values observed with the hydrogels may be attributed to the lower salts concentration and the presence of air inclusions trapped inside the hydrogel, whose presence cannot be avoided due to the function principle of the electrode cap and the increased hydrogel viscosity, in comparison to the conventional gel. Furthermore, a reduced skin hydration efficacy is expected for the hydrogels, as it was decided to add a mild skin penetration enhancer (Tween® 80) to the hydrogels, instead of more efficient components posing higher allergy risks [22,23]. Nevertheless, the hydrogel impedances are still well suited for EEG acquisition.

During an overall recording time of approx. 30 min, different EEG episodes were recorded including resting state EEG (eyes open), EEG with predominant alpha activity (eyes closed), and induced eye blinking and eye movement artifacts. Moreover, a visual evoked potential (VEP) test was recorded consisting of 300 checkerboard pattern reversal stimuli in accordance with the ISCEV 2010 standard. In FIG. 3, overlay plots of EEG are shown in the time domain. FIG. 3a shows EEG signals recorded with adjacent frontal channels LL1 and LD1, the former using conventional paste and the latter with hydrogel. These recordings contain externally triggered eye blink artifacts. FIG. 3b shows resting state EEG and alpha activity in exemplary recordings of channels LL13 and LL12, respective to the two electrolyte types (paste or hydrogel). The signal traces are very similar without considerable differences in both the signal shape and amplitude.

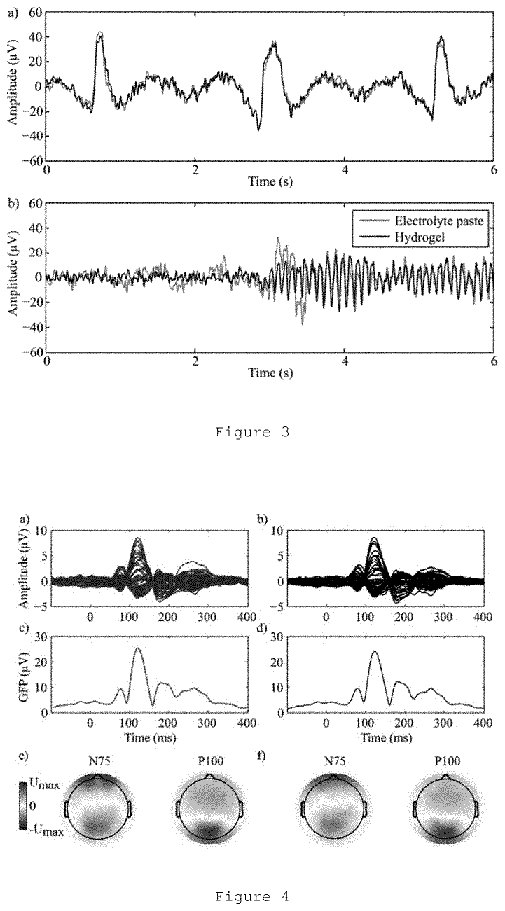

Very similar results were obtained for the grand average of the visual evoked potential (see FIG. 4). No substantial differences are visible in the individual channels (FIGS. 4a and 4b), in the global field power (GFP) (FIGS. 4c and 4d), nor in the exemplary topographic mappings of the N75 or P100 components (FIGS. 4e and 4f). The amplitudes, latencies and spatial potential distributions of the electrolyte and hydrogel signals are very similar.

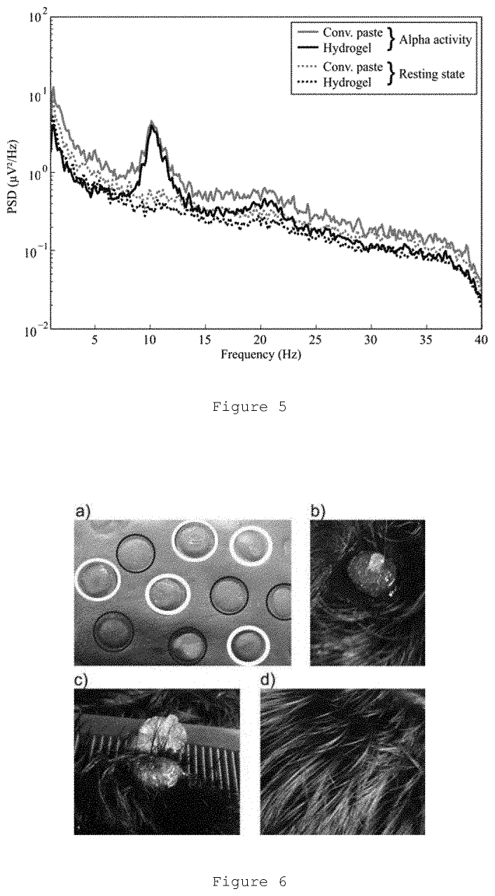

A similar result is visible in the frequency domain. FIG. 5 shows the mean Welch estimation of the power spectral density of EEG containing alpha activity (solid lines) and during resting state (dotted lines) for the frequency range of 1-40 Hz. The different spectra overlap each other for frequencies above 10 Hz. A slightly increased drift is visible for the commercial paste during the alpha activity tests, which may be related with paste running. However, this drift difference is less pronounced in the resting state EEG PSD. The alpha activity peak is clearly enhanced in the frequency range of 10-13 Hz for both the commercial paste and hydrogel.

Table II lists the quantitative results of the RMSD and CORR values (Pearson correlation coefficient) for the comparison between hydrogel and commercial paste for the different EEG tests. All values represent the mean and standard deviation (STD) over all subjects and channels. The results indicate a very good similarity of the compared EEG signals. According to our former studies [24-26], the differences evident in Table II can be caused by external noise and/or by the spatial distance of the compared adjacent electrodes on the volunteer's heads. Furthermore, the higher values of CORR and lower values of RMSD for the VEP are related to the increasing SNR due to the number of averaged stimulation epochs, as discussed next.

TABLE II
Quantitative EEG comparison results

| | Mean | Mean |
|---|---|---|
| | RMSD ± STD | CORR ± STD |
| EEG test | (μV) | (%) |
| Alpha | 5.1 ± 1.1 | 60.4 ± 7.2 |
| activity | | |
| Resting | 4.2 ± 0.8 | 58.5 ± 8.5 |
| state | | |
| Eyeblink | 5.6 ± 1.0 | 73.3 ± 8.9 |
| artifacts | | |
| VEP | 0.4 ± 0.2 | 86.4 ± 19.0 |

FIG. 6 shows photographs of the right fronto-temporal and CP1 head region of two volunteers. The photos were taken immediately after removing the EEG cap and are exemplary for all volunteers. Skin indentations indicate contact areas of the silicone cups of the cap, which generally disappear after a few minutes. It is clearly visible (FIG. 6a) that most hydrogel positions (black circles) are free of remnants, while all positions with conventional EEG paste (white circles) exhibit considerable amounts of residuals. The same observation can be made regarding the hairy positions of the head, since the hydrogel is easily removed with a comb (FIG. 6c) and the hair is completely clean after wiping for 10 s with a dry towel (FIG. 6d). Consequently, the cleaning effort of the subject's head after hydrogel application will be considerably reduced. This fact could be a great advantage in EEG acquisitions on patients with sensitive skin because it would reduce the overall stress on the scalp. As the product undergoes gelation within the predefined time after injection, no subsequent gel spreading or running is possible. Consequently, the risk of bridging adjacent electrodes and thus falsifying measurements is considerable reduced.

### BIBLIOGRAPHY

[1] M. Teplan, “Fundamentals of EEG measurement”, Meas. Sci. Rev. 2 (2002) 1-11.
[2] E. McAdams, “Bioelectrodes”, in Encyclopaedia of Medical Devices and Instrumentation, Webster J. G. Ed., New York, Wiley (1988) 120-166.
[3] S. Finnigan, S. Rose, M. Walsh, M. Griffin et al, “Correlation of Quantitative EEG in Acute Ischemic Stroke With 30-Day NIHSS Score”, Stroke, 35 (2004) 899-903.
[4] C. Plummer, S. Harvey, M. Cook, “EEG source localization in focal epilepsy: Where are we now?”, Epilepsia, 49(2) (2008) 201-218.
[5] T. Thompson, T. Steffert, T. Ros, J. Leach, J. Gruzelier, EEG Applications for Sports and Performance”, Methods, 45 (2008) 279.
[6] T. R. Mullen, C. A. E. Kothe, M. Chi, A. Ojeda, T. Kerth, S. Makeigg, T.-P. Jung, G. Cauwenberghs, “Real-Time Neuroimaging and Cognitive Monitoring Using Wearable Dry EEG”, IEEE Trans. Biomed. Eng., 62 (2015) 2553.
[7] M. Lopez-Gordo, D. Sanchez-Morillo, F. Pelayo Valle, “Dry EEG electrodes”, Sensors, 14(7) (2014) 12847-70
[8]-P. Griss, H. K. Tolvanen-Laakso, P. Meriläinen, G. Stemme, “Characterization of Micromachined Spiked Biopotential Electrodes”, IEEE Transs. Biom. Eng, 49(6) (2002) 597-604.
[9]-G. Ruffini, S. Dunne, L. Fuentemilla, C. Grau, E. Farrés, J. Marco-Pallarés, P. Watts, S. Silva, “First human trials of a dry electrophysiology sensor using a carbon nanotube array interface”, Sensors and Actuators A, 144(2) (2008) 275-279.
[10]-S. Kingsley, S. Sriram, A. Pollick, J. Marsh, “Photrodes for physiological sensing”, Optical fibers and sensors for biomedical applications IV, Proceedings of the Society of photo-optical instrumentation Engineers (SPIE), 5317 (2004) 158-166.
[11] V. Nikulin, J. Kegeles, G. Curio, “Miniaturized electroencephalographic scalp electrode for optimal wearing comfort”, Clin. Neurophysiology, 121 (2010) 1007-14.
[12] I. Volosyak, D. Valbuena, T. Malechka, J. Peuscher, A. Graser, “Brain-computer interface using water-based electrodes”, J. Neural Eng. 7 (2010) 066007.
[13] A. Searle, L. Kirkup, “A Direct Comparison of Wet, Dry and Insulating Bioelectric Recording Electrodes”, Physiol. Meas. 22 (2000) 71-83.
[14] C. Fonseca, J. P. Silva Cunha, R. E. Martins, V M Ferreira, J. P. Marques de Sa, M. A. Barbosa, A. Martins Silva, “A novel dry active electrode for EEG recording”, IEEE Trans. Biomed. Eng. 54(1) (2007) 162-165.
[15] see Waveguard cap from ANT, https://www.ant-neuro.com/products/waveguard_connect
[16] P. Fiedler, S. Griebel, P. Pedrosa, C. Fonseca, F. Vaz, L. Zentner, F. Zanow, J. Haueisen, “Multichanel EEG with novel Ti/TiN dry electrodes”, Sensors and Actuators A: Physical, 221 (2015) 139-147.
[17] R. Pasion, T. O. Paiva, P. Pedrosa, H. Gaspar, B. Vasconcelos, A. C. Martins, M. H. Amaral, C. Fernandes, J. M. Nóbrega, R. Pascoa, C. Fonseca, F. Barbosa, “Assessing a novel polymer-wick based electrode for EEG neurophysiological research”, Journal of Neuroscience Methods 267 (2016) 126-131.
[18] H. Peng, J. Liu, H. Tian, Y. Dong, B. Yang, X. Chen, C. Yang, “A novel passive electrode based on porous titanium for EEG recording”, Sensor Actuat B-Chem 226 (2106) 349-356.
[19] G. Li, D. Zhang, S. Wang, Y. Duan, “Novel passive ceramic based semi-dry electrodes for recording electroencephalography signals from the hairy scalp”, Sensors and Actuators B: Chemical, B 237 (2016) 167-178.
[20] N. Alba, R. J. Sclabassi, M. Sun, X. T. Cui, “Novel Hydrogel-Based Preparation-Free EEG Electrode IEEE T. Neur. Sys. Reh. (2010) 18, 415-423.
[21] K. Kleffner-Canucci, P. Luu, J. Naleway, D. Tucker, “A novel hydrogel electrolyte extender for rapid application of EEG sensors and extended recordings”, J. Neur. Methods, 206 (2012) 83-87.
[22] M. Lane, Int. J. Pharm. 2013, 447, 12.
[23] A. Williams, B. Barry, Adv. Drug Deliver Rev. 2012, 64, 128.
[24] P. Fiedler, J. Haueisen, D. Jannek, S. Griebel, L. Zentner, F. Vaz, C. Fonseca, ACTA IMEKO 2014, 3, 33.
[25] P. Fiedler, S. Griebel, P. Pedrosa, C. Fonseca, F. Vaz, L. Zentner, F. Zanow, J. Haueisen, Sensor Actuat. A-Phys. 2015, 221, 139.
[26] P. Fiedler, P. Pedrosa, S. Griebel, C. Fonseca, F. Vaz, E. Supriyanto, F. Zanow, J. Haueisen, Brain Topo. 2015, 28, 647.

1. Injectable hydrogel-forming polymeric composition that is capable of forming a hydrogel for reliable EEG monitoring and an easy scalp cleaning characterized by comprising: a first component, selected from the group consisting of natural and synthetic polymers; a second component, selected from the group consisting of a polymerization initiation system or a cross-linking agent.

2. Injectable hydrogel-forming composition according claim 1 characterized in that the first component is a solution comprising alginate, and the second component is a solution comprising calcium salts.

3. Injectable hydrogel-forming composition according to claim 1 characterized in that the first component further comprises at least one ionized salt in a concentration ranging from 0.1% to 10% to provide adequate electrical conductivity.

4. Injectable hydrogel-forming composition according to claim 1 characterized in that the first component further contains a humectant, preferably glycerol or propylene glycol, and a skin penetration enhancer, preferably Tween®80.

5. Injectable hydrogel-forming composition according to claim 1 characterized in that the first component is a solution comprising 2.8% (w/v) sodium alginate, 6% (v/v) Tween® 80, 10% (v/v) propylene glycol and 1.8% (w/v) sodium chloride; and the second component is a solution comprising 0.34% (w/v) calcium carbonate, 0.14% (w/v) calcium sulfate dehydrate and 1.18% (w/v) gluconolactone.

6. Hydrogel for reliable EEG monitoring and easy scalp cleaning, formed of the injectable hydrogel-forming composition as claimed in claim 1.

7. Hydrogel according to claim 6 characterized in that the gelation rate is adjustable by changing the alginate to calcium salts ratio.

8. Hydrogel according to claim 6 characterized in that the viscosity is adjustable by varying the alginate concentration.

9. Hydrogel according to claim 6 characterized in that it is suitable for application on the cavities of the EEG electrode.

10. Method of producing a hydrogel for reliable EEG monitoring and easy scalp cleaning, characterized by comprising: mixing the first component and the second component as claimed in claim 1.

11. Use of an injectable hydrogel-forming composition characterized by comprising the following steps:
i) providing a first component and a second component as claimed in claim 1;
ii) joining the first and second component to induce hydrogel production and applying into the electrode cavities of the EEG cap system;
iii) removing the EEG cap after EEG recording with attached solid hydrogel;
iv) cleaning the solid hydrogel pieces from the hair with a comb if necessary.

12. Use of an injectable hydrogel-forming composition according to claim 11 characterized in that in step ii) the first and the second component are mixed before application.

13. Use of an injectable hydrogel-forming composition according to claim 12 characterized in that in step ii) a double syringe equipped with a mixer nozzle is used to lower gelation time.
